(19) United States
(12) Patent Application Publication
(10) Pub. No.: US 2009/0285767 A1
(43) Pub. Date: Nov. 19, 2009

(54) USE OF OZONATED LIQUIDS AND PEROXIDES TO WHITEN TEETH

(76) Inventor: James E. Shenberg, Santa Monica, CA (US)

Correspondence Address: KOPPEL, PATRICK, HEYBL & DAWSON, 2815 Townsgate Road, SUITE 215, Westlake Village, CA, 91361-5827, US

(21) Appl. No.: 12/466,860

(22) Filed: May 15, 2009

Related U.S. Application Data
(60) Provisional application No. 61/053,424, filed on May 15, 2008.

(51) Int. Cl. A61K 8/22

(52) U.S. Cl. 424/53

(57) ABSTRACT

A method for the whiting of teeth includes the delivery of ozone dissolved in a fluid to the tooth surface. The ozone treatment can be followed by the application to the tooth surface of peroxide based tooth whitening compositions, combined with the peroxide whitener or delivered to the tooth surface coated with the peroxide whitening agent.

This application claims benefit of Provisional Application 61/053,424, filed May 15, 2008.

### BACKGROUND

Dental office procedures for whitening teeth are well known. Typically, the teeth are exposed for about 90 minutes to a tooth whitener comprising 18-35% hydrogen peroxide in a liquid or gel carrier.

In a typical procedure, a rubber sheet or dam is placed over the patient's teeth so that the teeth protrude through the sheet. The dam is made from latex rubber forced over each tooth and held in place with metal springs, clamps, and ligatures. This partially protects the soft tissues of the gums from the peroxides used in bleaching. However, since the rubber sheet stretches and does not custom fit the particular patient's mouth, the peroxide can leak around the rubber sheet and cause substantial discomfort to the patient. Typically, this method can be performed only on the upper or lower set of teeth at a time, not both. The composition is placed on the teeth and exposed to heat or a white light such as is generated by laser or halogen or zenon lights with a wavelength from about 380 nm to about 660 nm. The white light helps to release excited oxygen molecules which then interact with the tooth surface and the colored compounds in the tooth which cause a darkening color change in the tooth material. However, the majority of the active agents are released into the atmosphere rather than into the teeth and such techniques are therefore inherently inefficient. Further, exposure of the gums and oral mucosa to the peroxide treatment composition can result in pain for some time after the treatment.

Additionally, the dental office whitening procedures may be short lived. The whitening effect regress in the first month and studies have shown a loss of the whitening effect may commence in as few as seven days from completion of the in-office procedure.

Other tooth bleaching techniques may utilize both light and chemical activation of the hydrogen peroxide to accelerate the bleaching process. For example, Toh, C. G., “Clinical Evaluation of a Dual-activated Bleaching System,” Asian Journal of Aesthetic Dentistry, Vol. 1, No. 2, pp. 65 70 (July 1993), describes the use of a powder containing potassium persulphinate which is mixed with hydrogen peroxide solutions to form a paste. The paste was applied to discolored teeth and activated using a dental curing light. U.S. Pat. No. 5,032,178 to Cornell, discloses a 30-35% aqueous solution of hydrogen peroxide, with potassium persulphinate as an accelerator, and a dental curing light.

It is also known to use a laser in combination with hydrogen peroxide to bleach teeth. For example, U.S. Pat. No. 4,983,381 to Vicente M. Torres Zaragoza, generally discloses the use of laser radiation to accelerate the bleaching action of hydrogen peroxide.

U.S. Pat. No. 7,201,578 to Yarborough describes the use of light from an argon ion laser to activate bleaching agents applied to the teeth. The teeth are cleaned to remove any materials which may reduce or nullify the effects of the bleaching agents. A mixture of peroxide is applied to the teeth and the teeth are then exposed to light from the argon laser to activate the peroxide and accelerate the bleaching process.

Alternatively, boosters or catalysts can be used to further accelerate the bleaching process. Boosters, such as hydrogen and carbamide peroxide, decompose into free oxygen radicals, increasing the effectiveness of the bleaching compound beyond that of the hydrogen peroxide alone as a result of the increased free oxygen radical concentration.

U.S. Pat. No. 6,877,985 to Lynch describes the application of gaseous ozone to tooth surfaces for 10 to 20 seconds to whiten teeth. However, the patent includes no data regarding the efficacy of the treatment or a comparison with peroxide based treatment procedures. Using the device set forth in the Lynch '985 patent, Holmes et al. “Bleaching of Components Responsible for Extrinsic Tooth Discoloration by Ozone”, AADR Meeting (Mar. 13, 2003) describes the ability of gaseous ozone to whiten extrinsic discoloration as demonstrated by evaluation of the effect of ozone on brown-colored melanoidins which cause surface staining.

U.S. Pat. No. 7,244,354 to Burris et al describes the use of ozone dissolved in water or other solutions, such as isotonic saline, for treating teeth and for removing stains on teeth. However, Burris also does not provide any comparison of the efficacy of his treatment to peroxide treatments.

Domingo et al. reports that “Bleaching of Components Responsible for Tooth Discoloration by Ozone”, AADR 37^{th} Annual Meeting (Apr. 4, 2008) that ozonated water (5 ppm) generated using the TherOzone ozone generator (applicant's dissolved ozone delivery device) is effective in reducing extrinsic tooth discoloration cause by brown-colored melanoidins.

It is also known that combining hydrogen peroxide with ozone in solution, referred to as perozone, or exposing ozone to ultraviolet light increase the decomposition of ozone and increases the hydroxyl concentration in the solution rendering the solution and increases the efficacy of ozone for use in decontaminating water, for example in producing potable water from waste water or ground water, and decontaminating soil (EPA Guidance Manual, Alternative Disinfectants and Oxidants, Chapter 7, “Peroxone (Ozone/Hydrogen Peroxide), April 1999). However, there has been no suggestion to use such a composition for dental applications or for tooth whitening.

It would be advantageous to provide a tooth whitening technique that requires a lower concentration of hydrogen peroxide in the whitener to reduce pain, a shorter exposure of the teeth to the whitening agent and an increased efficacy and longevity of the whitening effect.

### SUMMARY

The present invention generally relates to a method for whitening teeth, and more particularly, relates to a method for whitening teeth which utilizes solutions or gels containing dissolved ozone as a tooth pretreatment prior to applying peroxide whitening agents. Alternatively, ozone can be dissolved in a peroxide solution to produce a more effective tooth whitening agent. Where prior uses of gaseous or dissolved ozone alone to whiten teeth have a superficial effect and are primarily useful for removing bacteria and surface stains (extrinsic discoloration), by using the combination described herein intrinsic discoloration, that is discoloration which is within the tooth and not merely on the surface, is addressed and a superior whitening effect can be accomplished in a shorter period of time. This whitening effect is longer lasting and the combination of dissolved ozone followed by and oxidative treatment provides more then just an additive effect. The need for external light, heat or oxygenation activators can also be reduced or eliminated.

### DETAILED DESCRIPTION

White teeth have long been considered cosmetically desirable. Typically, a tooth becomes discolored in specific regions, or spots, and in overall color. Tooth materials which are generally responsible for presenting a stained appearance are enamel, dentin, cementum and the acquired pellicle.

Tooth enamel is formed predominantly from inorganic material, mostly in the form of hydroxyapatite crystals, containing approximately 5% organic material, primarily in the form of collagen. Dentin is composed of about 20% protein, including collagen, with the balance being inorganic material, predominantly hydroxyapatite crystals. On the other hand, the pellicle is a proteinaceous layer which forms on the surface of tooth enamel, and rapidly reforms following removal using intensive tooth cleaning with highly abrasive prophylaxis pastes.

Tooth discoloration results from both extrinsic and intrinsic staining. Extrinsic staining of the tooth surface arises as a result of the accumulation of various chromogenic substances and initially colorless chromogen precursors which convert to chromogens within the pellicle that forms on the tooth surface. This type of staining can usually be removed by mechanical methods, which remove the acquired pellicle or portions thereof, along with the adherent chromogens.

As the extrinsic stains age they become less susceptible to removal by mechanical means which may be due to penetration of the extrinsic stain into the enamel over a period of time. As a result such stains, therefore, require the use of chemicals, such as oxygenating agents, which can penetrate the tooth enamel to oxidize or solubilize the deep-seated chromogens.

In contrast, intrinsic staining occurs as a result of chromogenic substances derived from sources within the tooth and can not be removed by mechanical tooth cleaning methods. In addition, tooth filling materials can contribute to variation in coloration density resulting in darkened areas.

Abrasive erosion of stained acquired pellicle using liquids, gels, or pastes including toothpastes, that are mechanically agitated at the stained tooth surface are generally used to remove or reduce extrinsic tooth staining. To address intrinsic discoloration chemical containing liquids, gels, or pastes are applied to the tooth surface for a specific period, after which the formulation is removed. In some cases the mechanical process is supplemented by an auxiliary chemical process, which may be oxidative or enzymatic. Care must be taken to protect the soft tissue of the gums from peroxides typically used in the bleaching procedure.

In accordance with a first embodiment the present invention, the teeth to be whitened are first exposing to ozone dissolved in a liquid and then peroxide based whitening agents are applied to the ozone treated teeth. For ease of discussion the ozone dissolved in a liquid will be referred to as “ozonated water”. However, such termination is not intended to limit the scope of the invention as the ozone may be dissolved in various different liquids, such as saline solutions or oil or water based gel carriers. Preferably, the teeth are abrasively cleaned prior to applying the ozonated water. The method may be applied over a number of teeth or an individual tooth. Further, as hereinabove noted, portions of a tooth may also be whitened in accordance with the method of the present invention.

One skilled in the art will recognize that numerous different devices and techniques may be used to apply the ozonated water to the tooth surface. For example, a delivery device such as shown by Lynch in U.S. Pat. No. 6,877,985, incorporated herein in its entirety by reference, for delivering gaseous ozone may be used. Alternatively, a liquid delivery device such as a WATER-PIK® or dental office irrigation and aspiration system can be used. Still further, electric tooth brushes which include vibrating bristles or a rotating abrasive head can be provided with a fluid delivery port so that the ozonated water can be delivery while the tooth surface is being abrasively cleaned.

The ozonated water is delivered at a pressure, concentration and for a period of time sufficient to provide removal of extrinsic discoloration of tooth enamel. The upper limit on ozone concentration in the solution is determined by safe patient and dentist exposure levels. Preferred concentrations are from about 3 to about 10 ppm with a more preferred concentration being from about 3 to 6 ppm. In one embodiment the ozonated water is used as a mouthwash for 2 minutes prior to a standard peroxide treatment.

Besides addressing extrinsic discoloration, the ozone aids in removing the pellicle, opens the pores within the tooth and penetrates into the enamel. This action appears to render the intrinsic discoloration more amenable to the action of the peroxide based whitening treatment, possibly by enhancing peroxide penetration and opening and activating the surfaces of the pores within the teeth. Some of the ozone may remain within the tooth and in turn promote the initiation of peroxide breakdown and the release of superoxide and hydroxyl free radicals as well as adjust the pH within the tooth to, or closer to, 11.8 which is the optimum pH for the peroxide to effect color removal. The ozone when used in the described combination may also attack intrinsic discoloration.

Based on initial patient trials, when compared with the use of the peroxide treatments alone, the combination of ozonated water followed by peroxide whiteners results in a faster treatment, brighter, whiter teeth and a longer lasting whitening effect. Patients appear to have a reduced tooth sensitivity following the procedure then with standard peroxide treatments alone, lower concentrations of peroxide are required to obtain comparable or better results and the whitening procedure can be accomplished in a shorter period of time. Also less exposure to laser, UV light or heat may be required to accelerate the whitening process, or use of the same level of exposure may result in an enhanced whitening effect. As an added benefit, if a two minute ozonated water mouth wash is first used it has been discovered that up to about a 6% by volume peroxide solution can be used without a rubber dam, gum protective gels or other common techniques used to protect the gingivae. Apparently the pretreatment with ozonated water provides some protective action to the tissue later exposed to peroxide and there is less incidence of subsequent gum irritation and more rapid healing. This may be a result of a reduction in bacteria within the mouth as a result of the ozone treatment, thus preventing or reducing subsequent gum tissue inflammation.

This effect is more than just additive. Ozone has been shown to attack extrinsic discoloration, but has not been demonstrated to address intrinsic discoloration. Peroxides address intrinsic discoloration and provide a known level of whitening. The use of ozonated water followed by peroxide treatment significantly increases the efficacy of the peroxide treatment as well as reduces the time necessary to generate the desired effect.

In a second embodiment ozone dissolved in a liquid or gel is combined with a peroxide based tooth whitening agent. This combination can then be applied to the teeth to be whitened in the same manner as prior whitening agents to provide a faster acting whitening agent with better whitening of the intrinsic discoloration of the teeth. Still further, the peroxide application can be followed by delivery of the ozonated water to the peroxide coated tooth surface.

In still further embodiments, a tooth whitening agent for removal or reduction of intrinsic tooth discoloration can be produced by using agents other then peroxide to decompose the ozone, releasing hydroxyl free radicals, or these other agents can be combined with the peroxide to release hydroxyl free radicals or superoxide free radicals. For example, these decomposition promoters can include ions of Cr, Zn, V, W, Fe, Mo, Ni, Co, Ru, Cu, Rh, Pd, Ag or Pt, oxides of these metals, such as MnO₂, Fe₂O₃, Ag₂O, NiO or CuO, or multicomponent compositions, such as MnO₂—TiO₂, MnO₂-alkali metal or alkaline earth metal oxides. These agents could be added to the ozone composition or applied to the teeth prior to treatment with the ozone and peroxide compositions to act as initiators or promoters of the reaction. In a still further embodiment these agents could be applied to the teeth during the same whitening procedure but after the oxidative whitening agent has been applied to activate or decompose the peroxide or to adjust the pH of the tooth closer to or to a pH of 11.8. An example would be the addition of an iron based compound or an alkali compound. It should be recognized that the higher the pH or the temperature of the environment the faster ozone will decompose and release the hydrogen free radicals which are effective in decomposing the compounds within the teeth that result in discoloration.

While the procedures described herein are directed to the use of ozonated water, it is also contemplated that the unique compositions described herein and the superior whitening effects can be obtained using gaseous ozone in place of the ozonated water.

I claim:

1. A process for whitening teeth comprising:
exposing the teeth to be whitened to ozonated water to provide ozone-treated teeth and exposing the ozone-treated teeth to a peroxide based whitening agent.

2. The process of claim 1 further including abrasively cleaning the teeth prior to exposing the teeth to ozonated water.

3. The process of claim 1 wherein sufficient time is provided for the ozonated water to remove the extrinsic discoloration from the tooth before the peroxide based whitening agent is applied.

4. The process of claim 1 wherein the ozone concentration in the ozonated water is from about 3 to about 10 ppm.

5. The process of claim 1 wherein the ozone concentration in the ozonated water is from about 3 to 6 ppm.

6. The process of claim 1 wherein the ozonated water is used as a mouthwash for two minutes prior to applying the peroxide based whitening agent.

7. The process of claim 6 wherein the peroxide concentration is less than about 6%ᵥ.

8. The process of claim 1 wherein sufficient time is provided for the ozonated water to remove the extrinsic discoloration from the tooth and to adjust the pH in the tooth to about 11.8 before the peroxide based whitening agent is applied.

9. A process for removing extrinsic discoloration and intrinsic discoloration from teeth comprising exposing the teeth to ozonated water followed by a peroxide tooth whitening composition.

10. A process for removing extrinsic discoloration and intrinsic discoloration from teeth comprising dissolving ozone in a liquid or gel to form an ozone composition combining the ozone composition with a peroxide based tooth whitening agent to form an ozone-peroxide whitening agent and applying the ozone-peroxide whitening agent to the teeth to be treated.

11. A process for removing extrinsic discoloration and intrinsic discoloration from teeth comprising applying a peroxide based tooth whitening agent to the tooth surface and applying ozonated water to the tooth surface which is peroxide coated.

12. A process for removing extrinsic and intrinsic discoloration from teeth using a peroxide whitening agent comprising adding one or more compounds containing ions of Cr, Zn, V, W, Fe, Mo, Ni, Co, Ru, Cu, Rh, Pd, Ag or Pt, or oxides thereof to ozonated water before applying the ozonated water and peroxide whitening agent to the teeth or applying one or more compounds containing ions of Cr, Zn, V, W, Fe, Mo, Ni, Co, Ru, Cu, Rh, Pd, Ag or Pt, or oxides thereof to the tooth surface prior to applying the ozonated water and peroxide whitening agent to the teeth.
